(19) United States
(12) Patent Application Publication
(10) Pub. No.: US 2025/0067701 A1
(43) Pub. Date: Feb. 27, 2025

(54) ELECTROCHEMICAL TYPE BIOSENSORS COMPRISING RESORCARENES

(71) Applicant: BIOSENSING S.R.L., Roma (IT)

(72) Inventors: Bruno BOTTA, Rome (IT); Francesca GHIRGA, Rome (IT); Deborah QUAGLIO, Rome (IT); Andrea CALCATERRA, Rome (IT); Cristina DEL PLATO, Rome (IT); Franco MAZZEI, Rome (IT); Francesca POLLI, Rome (IT)

(21) Appl. No.: 18/723,277

(22) PCT Filed: Jan. 26, 2023
(86) PCT No.: PCT/IB2023/050685

§ 371 Date: Jun. 22, 2024

(30) Foreign Application Priority Data
Jan. 28, 2022 (IT) 102022000001490

(51) Int. Cl. G01N 27/327; C07C 59/72; C07C 69/712; G01N 27/30; G01N 33/53; G01N 33/543

(52) U.S. Cl. CPC G01N 27/3278; C07C 59/72; C07C 69/712; G01N 27/308; G01N 27/3276; G01N 33/5308; G01N 33/5438; C07C 2603/92

(57) ABSTRACT

The present invention relates to novel resorc[4]arenes, such as pre-organized structure linkers for the development of high performance electrochemical biosensors.

### FIELD OF THE INVENTION

The present invention concerns novel compounds, such as pre-organized structure linkers for the development of high performance electrochemical biosensors.

### BACKGROUND ART

The size of the global biosensors market in 2020 was estimated to be approximately USD 22.4 billion, with an expected expansion at a compound annual growth rate (CAGR) of 7.9% from 2021 to 2028. Biosensors, thanks to their ability to assess the state of health, the onset and the progression of diseases, will be widely used by National Health Systems to monitor the condition of patients at home (telemedicine) stimulating the growth of the market.

The home health diagnostics segment should be characterized by the highest CAGR equal to approximately 10.4% in the 2021-2028 period. Key factors attributing this growth include the increasing prevalence of diseases such as diabetes, cardiovascular disease and cancer. The increasing demand for inexpensive and reliable sensors for routine monitoring of patients at home and the technological advances in the development of new products capable of delivering fast, inexpensive and accurate results should help increase the rate of use over the period considered. In addition, the considerable technological advances have made it possible to extend the use of this technology to other sectors, for example the agricultural applications should be characterized by a higher CAGR by 9.6% from 2021 to 2028. Biosensors, in fact, allow the rapid and specific detection of contaminants to reduce damage to farms and crops. These devices can also be used to determine the concentration of herbicides, pesticides, heavy metals and other pollutants in water and soil.

Among the various biosensors, electrochemical ones still account for the largest market share with a turnover of more than 70% in 2020, thanks to their peculiar characteristics such as miniaturisability, simplicity, low costs, speed of measurement and sensitivity.

Among the major industries involved in the biosensors market we can mention: Abbott (US), Roche (Switzerland), Medtronic (Ireland), Bio-Rad Laboratories, Inc. (US), DuPont (US), Biosensors International Group, Ltd. (Singapore), Cytiva (UK), Dexcom, Inc. (US), Lifescan IP Holdings, LLC (US), Masimo (US), Nova Biomedical (US), Universal Biosensors (Australia), Cranfield Biotechnology Centre (UK).

To date, particular attention has been paid to immunosensors, which represent an important diagnostic tool in several areas, such as for example clinical and food area. In recent years, research has focused on improving this technology, in terms of selectivity, sensitivity, rapidity in response and miniaturization, aimed at monitoring and screening analytes present in different matrices. One of the main limitations in the development of high-performance immunosensors concerns the immobilization of the antibody on the electrode surface. Recently, increasing interest has been directed to the possibility of applying calixarenes, cyclic oligomers, as linkers in the development of biosensors, in order to improve the orientation of biomolecules (e.g., enzymes, proteins, antibodies) and, therefore, to optimize the response of the sensor to a specific analyte. In the literature, differently modified calixarenes have been used in the development of biosensors: i) tetra-tert-butyl(3-thiol-1-oxypropane)dihydroxy calixarene has been used for the surface modification of gold electrodes, favouring the formation of self-assembled monolayers (SAMs) for the immobilization of the enzyme glucose oxidase (Demirkol D. O. et al. 2014); ii) the calixarene derivative, functionalized in both the upper and lower rim with primary amino groups, has been used for the surface modification of montmorillonite, favouring the immobilization of the enzyme pyranose oxidase (Sonmez B. et al. 2014); iii) calixarenes, functionalized in the lower rim respectively with esters, carboxylic acids and crown ethers (e.g., Prolinker™) and in the upper rim with thiol groups, have been used as linkers in the oriented immobilization of different antibodies and proteins such as, for example, anti-chorionic gonadotropin Ab, immunoglobulins G, integrins (Chen H. et al. 2008a; Lee Y. et al. 2003; Soler M. et al. 2014); iv) recently, compounds with a resorcarene structure have been used as linkers for the realization of optical immunosensors, with the aim of favouring the correct orientation of anti-insulin antibodies (Quaglio D. et al. 2020). To promote interaction with the antibody, several functionalizations of the upper rim have been described in the literature, while thio-alkyl chains in the lower rim have allowed the formation of a homogeneous SAM layer on the gold surface, increasing the number of linker binding sites and decreasing unwanted interactions.

Among the examples reported, some envisage the immobilization of biomolecules by forming covalent bonds that, therefore, make it impossible to regenerate the functionalized surface of the sensor; other systems, on the other hand, have the advantage of using non-covalent interactions in the bond with the biomolecule, but the disadvantage of possessing poor solubility in aqueous, non-toxic and biocompatible solvent, limiting their use for the development of electrochemical immunosensors.

To date, in fact, there are few examples of patents concerning the use of calixarenes as artificial linkers and only Prolinker™ has been marketed by the company Proteogen, Inc. (Korea) for the development of biosensors (Chen H. China Patent No. CN109342529B, 2008b; Jung Sung Ouk and Lee Soo Suk. Korean Patent No. KR100773543B1, 2007; Kim T-S. Patent No. EP1110964A1; 2002). WO03107007 describes a rapid test method for the detection of at least one antigen by optical and/or chemical detection, based on the use of antibody-linked calixarenes or resorcarenes and wherein the quantification of a possible antigen is carried out with methods mostly of colorimetric type. In the specific case of resorcarenes, only one article reports their possible use as artificial linkers for the development of immunosensors (Quaglio D. et al. 2020).

Therefore, the design of pre-organized linkers for the development of high-performance electrochemical immunosensors appears urgent.

### SUMMARY OF THE INVENTION

In recent years, scientific and industrial interest in molecules designed as linkers for the development of high-performance electrochemical immunosensors has been growing.

A unique feature of immunosensors is the coupling of the antibody to the chemical-physical signal transducer through chemical or physical immobilization techniques. Most of the immobilization methods used are characterized by the random orientation (random immobilization) of the antibody (Ab) with consequent reduction of the sites available for interaction with the antigen (Ag) and, therefore, of the sensitivity of the biodevice. The immobilization procedure is usually divided into two steps: i) modification of the sensor surface using SAM, functionalization of nanomaterials, electropolymerization, etc. . . . ; ii) binding with Ab through crosslinking reactions, oriented immobilization, adsorption, entrapment in the polymer. Taking these premises into account, the need for innovative solutions capable of optimising the antibody immobilization process on the sensor surface for the development of high-performance immunosensors is therefore evident. In this context, supramolecular chemistry represents an important tool for directing the immobilization of the antibody on the electrode surface according to an end-on orientation, by using pre-organized synthetic receptors that allow to optimize the functional properties of the SAM. Among the different types of macrocycles available, calixarenes have shown good versatility in site-specific immobilization of the antibodies in the development of immunosensors. In this regard, the aim of this invention has been to realize electrochemical immunosensors with optimized performance based on the use of resorcarenes, belonging to the calixarene family and, within the calixarene family, characterized by greater conformational flexibility and chemical versatility. Such properly functionalized macrocycles in the upper and lower portions are capable of forming both a compact SAM on the gold surface of magnetic nanoparticles deposited on the electrochemical sensor, and of interacting with the Fc fragment of the antibody (Šustrová B. et al. 2010).

The present invention will now be illustrated with non-limiting examples with reference to the following figures.

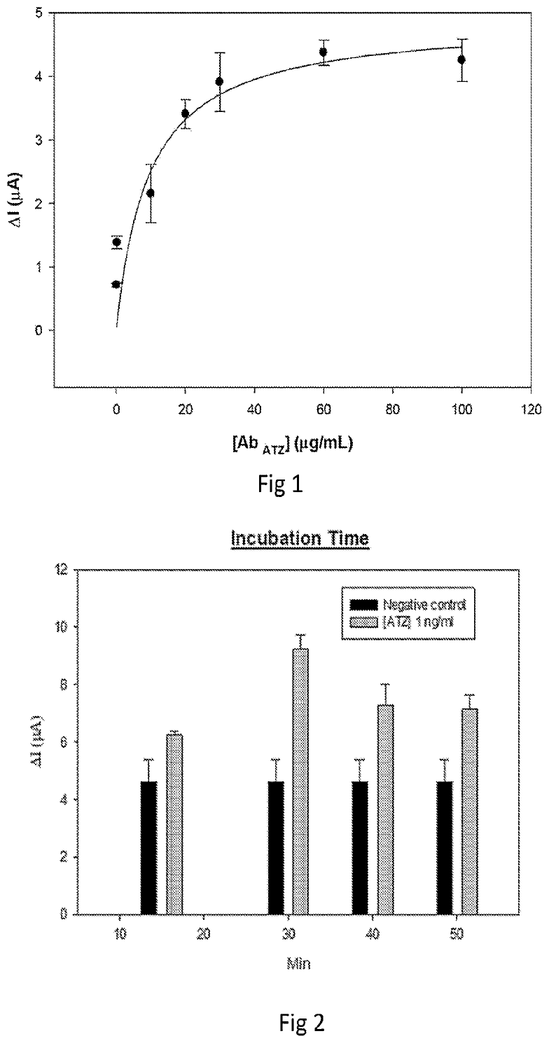

FIG. 1. Electrode current intensity variations following deposition of Ab-ATZ at different concentrations.

FIG. 2. Histogram relative to the values of Δi (μA) due to the different ATZ incubation times (1 ng/ml).

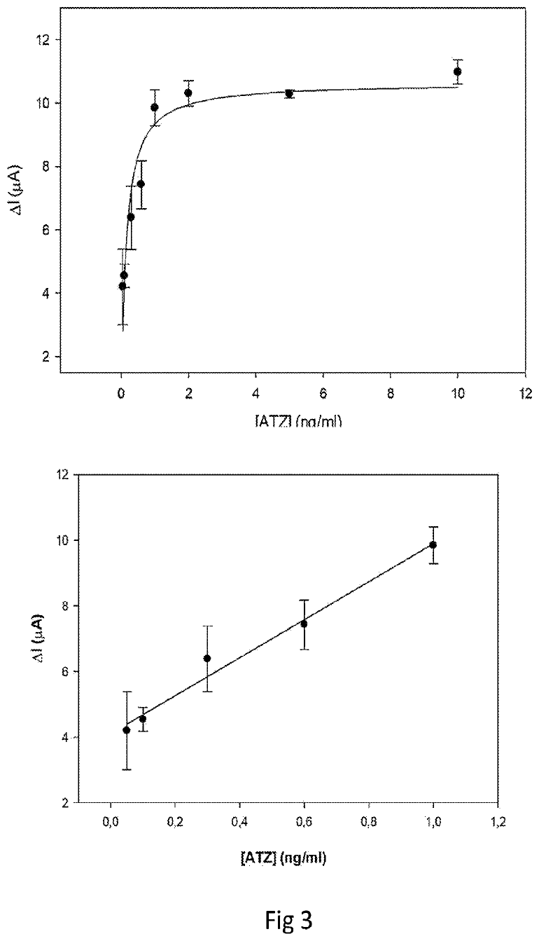

FIG. 3. ATZ adsorption isotherm and sensor calibration straight line in the range 0.05-1 ng/mL.

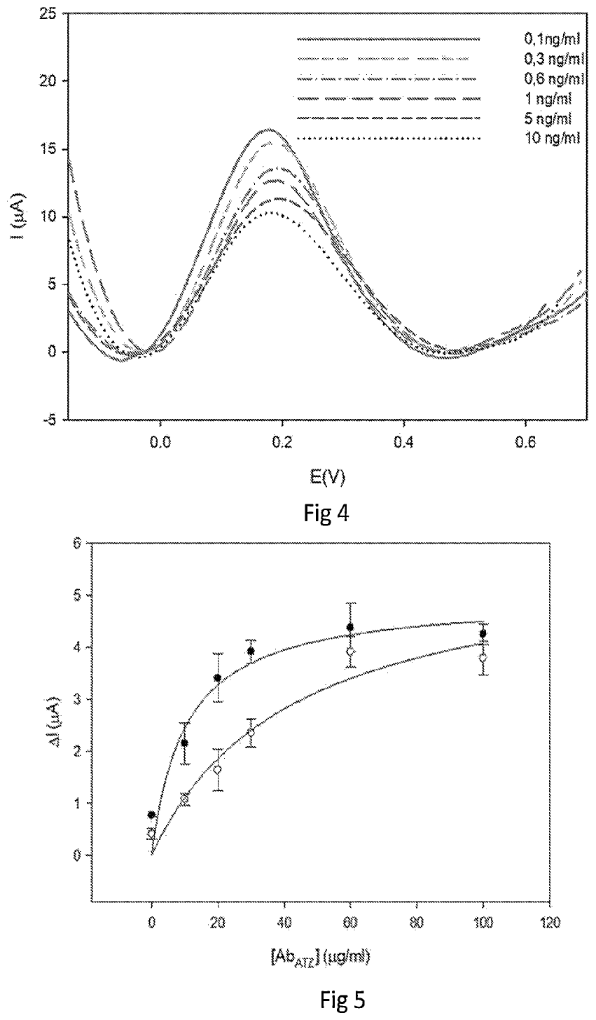

FIG. 4. DPV signals relative to the interaction between the immunosensor and ATZ standard solutions.

FIG. 5. Comparing AbATZ adsorption isotherms relative to oriented immobilization (in black) and randomization (in white).

### DETAILED DESCRIPTION OF THE INVENTION

The authors of the present invention have developed a resorc[4]arene linker, able to direct the Abs, with the advantage of increasing the quantity immobilized while maintaining the ability to bind specifically with the Ags, allowing to increase the sensitivity of the measuring device. In order to obtain a good oriented immobilization of the Abs and, in the same way, extend the use of artificial receptors to screen printed gold electrodes (SPE), thus allowing the miniaturization of the measuring device, the compounds object of the present patent application, represented for example by the compound identified with the acronym RW, have been designed and synthesized.

The linker compounds of the invention are macrocyclic systems belonging to the resorcarene family. These cyclic oligomers, derived from resorcinol, are among the classes of compounds most used in supramolecular chemistry as host species for molecular recognition of host species. The resorcarenes have a unique three-dimensional structure, characterized by a large central cavity, which can be chemically modified in the upper and lower rims with different functional groups. In the present invention, the rational design of resorc[4]arene linkers, such as for example the RW compound, has provided for:
I) the introduction of polar groups in the upper rim, with the aim of favouring interaction with specific amino acid residues of the Fc portion of the antibody in the end-on configuration;
II) the functionalization of the lower rim with alkyl chains containing thioether groups, functional to the anchoring on the surface of gold electrodes through the formation of the SAM;
III) the introduction of negatively charged groups, capable of favouring solubility in aqueous, non-toxic and biocompatible solvent, in order to avoid degradation of SPEs.

In particular, the RW linker, thus designed and synthesized, turned out to be able to bind the Fc portion of different Abs by non-covalent interactions, e.g., dipole-dipole and electrostatic, allowing the possible regeneration of the functionalized surface after the measurement of the Ag-Ab interaction. One of the main advantages in using the RW compound for the development of high-performance immunosensors consists in combining the correct immobilization of the antibody with the possibility of developing electrochemical sensors.

In one embodiment the present invention concern compounds of formula (I), such as artificial linkers capable of directing site-specific immobilization of antibodies according to an end-on orientation, wherein:

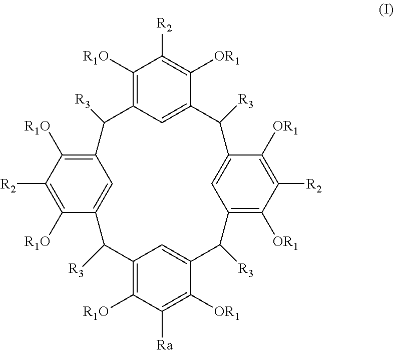

R₁ is selected from C₂₋₆alkyl, (CH₂)ₙC(═O)OX (wherein 1≤n≤5), SO₃X, PO(OX)₂, (CH₂)₂OH;
wherein X are positively charged atoms or groups comprising: Na⁺, K⁺, NH₄+;
R₂ is selected from hydrogen, [heterocycle]-C(═O)OX, CH₂-[heterocycle]-C(═O)OX, CH₂SO₃X, CH₂PO(OX)₂, wherein X are positively charged atoms or groups such as: Na⁺, K⁺, NH₄⁺; preferably said heterocycle is piperidine, pyrrolidine or piperazine;
R₃ is selected from
   a saturated linear aliphatic group containing sulfur of molecular formula (CH₂)ₘSH (5≤m≤11) or (CH₂)ₘS(CH₂)ₘCH₃ (5≤m≤11), or
   an aromatic group of molecular formula C₆H₄—SH, C₄H₃S or C₆H₄—N₂Y wherein Y are negatively charged atoms or groups comprising: Cl⁻, BF₄⁻.

As used herein, the terms “heterocycle” or “saturated heterocycle”, used interchangeably, mean a 4-7 term saturated cyclic compound comprising at least one heteroatom; examples of heterocycles are, for example, azetidine, pyrrolidine, piperidine, piperazine, morpholine, thiomorpholine, azacycloheptane; preferably the saturated heterocycle is piperidine, pyrrolidine or piperazine, preferably linked via the nitrogen atom, directly or via a methylene bridge, to the aryls of the compound of general formula (I) or of general formula (II), as will be described below.

As used herein, (CH₂)ₙC(═O)OX indicates an aliphatic chain comprising n methylene units (CH₂), functionalized at one end with a carboxyl group C(═O)OX, wherein X is the positively charged counterion balancing the negative charge of the carboxylate, and bonded at the other end to the reference scaffold. Thus in the present invention, (CH₂)ₙC(═O)OX indicates for example —CH₂C(═O)OX, —CH₂CH₂C(═O)OX, —CH₂CH₂CH₂C(═O)OX etc., wherein X is the positively charged counterion balancing the negative charge of the carboxylate. Similarly, (CH₂)ₘSH indicates an aliphatic chain comprising m methylene units (CH₂), functionalized at one end with an SH group and linked at the other end to the reference scaffold; (CH₂)₂OH indicates an aliphatic chain comprising two methylene units (CH₂), functionalized at one end with an OH group and linked at the other end to the reference scaffold. The terms CH₂-[heterocycle]-C(═O)OX, CH₂SO₃X, CH₂PO(OX)₂ indicate methylene groups linked to the reference scaffold and to the indicated functional group.

A preferred embodiment of the invention subject-matter of the present description concerns a compound of general formula (I) wherein: R₁ is (CH₂)ₙC(═O)OX, preferably CH₂C(═O)OX, and/or R₂ is hydrogen and/or R₃ is a saturated linear aliphatic group containing sulfur of molecular formula (CH₂)₁₀S(CH₂)₁₁CH₃; in a further preferred embodiment X is NH₄⁺; in a further preferred embodiment R₁ is CH₂C(═O)OX, R₂ is hydrogen, R₃ is a saturated linear aliphatic group containing sulfur of molecular formula (CH₂)₁₀S(CH₂)₁₁CH₃ and X is NH₄⁺.

In a further embodiment, the invention concerns an electrode for the electrochemical detection of an analyte in a biological sample, wherein the electrode is a printed miniaturized carbon-based electrode comprising magnetic nanoparticles, preferably coated with gold, said nanoparticles comprising on their surface the compound of formula (I) as defined above; preferably said printed miniaturized carbon-based electrode is a graphite, graphene, carbon nanotubes or carbon fibers electrode; in a further preferred embodiment the compound of formula (I) is non-covalently linked to at least one antibody able to detect to said analyte.

Gold-coated magnetic nanoparticles (Au@MNPs) are currently used primarily for the bioseparation and the development of electrochemical and optical sensors, for the preparation of contrast agents for magnetic resonance imaging, or for the targeted delivery of drugs. The gold-coated magnetic nanoparticles used herein are commercially available or can be obtained by procedures described in the literature (Chem. Commun., 2016, 52, 7528-7540).

In a further embodiment, the invention concerns a biosensor for the electrochemical detection of an analyte in a biological sample comprising the electrode as above defined as a working electrode, a reference electrode and a counter electrode.

In a preferred embodiment, the biosensor for the electrochemical detection of an analyte in a biological sample comprises:
a printed miniaturized carbon-based electrode comprising magnetic nanoparticles; preferably said electrode is a graphite, graphene, carbon nanotubes or carbon fibers electrode;
the compound of formula (I) as defined above non-covalently linked with at least one antibody capable of recognizing the analyte.

In a further preferred embodiment, the magnetic nanoparticles are gold-coated magnetic nanoparticles modified on the surface with the compound of general formula (I).

In a further preferred embodiment, in the biosensor for the electrochemical detection of an analyte in a biological sample, the compound of formula (I) non-covalently binds at least one antibody capable of recognizing atrazine (ATZ).

In a further embodiment, the present invention comprises a method for the production of the biosensor as defined above for the electrochemical detection of an analyte in a biological sample comprising the following steps:
(a) adding magnetic nanoparticles preferably coated with gold to a solution comprising the compound of formula (I), such that at least one compound of formula (I) is bound on the surface of the nanoparticles;
(b) depositing the resulting solution on the surface of a printed miniaturized carbon-based electrode and evaporating the liquid phase of the solution, preferably in the presence of a magnet located below the electrode;
(c) adding to the electrode surface a solution comprising at least one antibody specific for an analyte obtained at the end of step (b) and incubating for sufficient time for the formation of a non-covalent bond with the compound of formula (I).

Preferably, in step (a) the compound of formula (I) is used in aqueous solution and in a concentration comprised between 1 μM and 4 mM; again preferably, in step (c) the antibody is used in a concentration comprised between 0.1 and 100 μg/ml.

In a further embodiment, the present invention concerns a method for the electrochemical determination of an analyte in a biological sample comprising the following steps:
a) contacting the biological sample with the biosensor as defined above, so that the antibody present on the working electrode binds the analyte resulting in a change in the passage of current between the working electrode and the counter electrode;
b) detecting the current change generated in step a);
c) measuring the presence and/or the quantity of the analyte in the sample by comparison with a calibration curve.

In a further embodiment, the invention concerns an electrode for the electrochemical detection of an analyte in a biological sample, wherein the electrode is a printed miniaturized carbon-based electrode comprising magnetic nanoparticles, preferably coated with gold, said nanoparticles comprising on their surface the compound of formula (II):

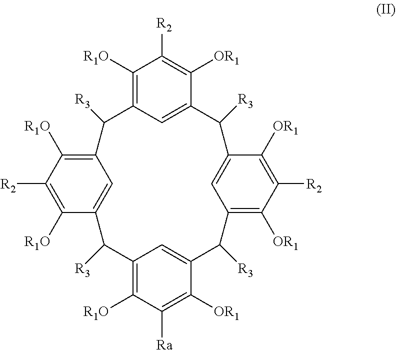

Wherein
R₁ is selected from hydrogen, alkyl, (CH₂)ₙC(═O)OX (1≤n≤5), SO₃X, PO(OX)₂, (CH₂)₂OH, wherein X are positively charged atoms or groups comprising: Na⁺, K⁺, NH₄⁺;
R₂ is selected from hydrogen, [heterocycle]-C(═O)OX, CH₂-[heterocycle]-C(═O)OX, CH₂SO₃X, CH₂PO(OX)₂ wherein X are positively charged atoms or groups such as: Na⁺, K⁺, NH₄⁺; preferably the heterocycle is piperidine, pyrrolidine or piperazine;
R₃ is selected from
   a saturated linear aliphatic group containing sulfur of molecular formula (CH₂)ₘSH (5≤m≤11) or (CH₂)ₘS(CH₂)ₘCH₃ (5≤m≤11);
   an aromatic group of molecular formula C₆H₄—SH, C₄H₃S, C₆H₄—N₂Y wherein Y are negatively charged atoms or groups, preferably Cl⁻, BF₄⁻;
wherein said compound of formula (II) non-covalently binds at least one antibody capable of recognizing said analyte.

The invention concerns the use of said electrode as a working electrode in a biosensor for the electrochemical detection of an analyte in a biological sample, said biosensor further comprising a reference electrode and a counter electrode.

Overall, the data of the invention in question propose a new artificial linker, such as in particular the RW compound, capable of directing the site-specific immobilization of antibodies according to an end-on orientation, and emphasize the effectiveness of the compound as a new potential linker capable of increasing the performance of electrochemical immunosensors.

### Examples

The following examples are reported for illustrative purposes only and are not intended to limit the scope of the present invention. Variations and modifications of any of the embodiments described herein, which are obvious to a person skilled in the art, are encompassed by the scope of the appended claims.

### Rational Drawing and Synthesis of the RW Compound

### Chemicals, Reagents and Methods of Analysis

All reagents and solvents are commercially available and have been used without further purifications.

Silica gel (230-400 mesh) was used for purification by flash column chromatography. All reactions were monitored by thin layer chromatography (TLC) and F254 fluorescence silica gel plates (Sigma-Aldrich 99569) were used. The melting points were determined with a Buchi Melting Point B-454. The 1H and 13C NMR spectra were recorded with a Bruker 400 Ultra Shield™ instrument (400 MHz for ¹H NMR and 100 MHz for ¹³C NMR), using tetramethylsilane (TMS) as a standard. Chemical shifts are reported in parts per million (ppm). Multiplicities were reported as follows: singlet (s), doublet (d), triplet (t) and multiplet (m). Mass spectrometry was performed with the Thermo Finnigan LXQ linear ion trap mass spectrometer, equipped with electrospray ionization (ESI). High-resolution mass spectra (HRMS) were recorded with a Bruker BioApex Fourier transform ion cyclotron resonance (FT-ICR).

### Synthesis Procedures

Synthesis of compound 2: 10-undecen-1-ol (MERCK 203-971-0) (41.11 mmol, 7 g) was added to a solution of pyridinium chlorochromate (MERCK 247-595-5) (61.66 mmol, 13.3 g) and celite (MERCK 272-489-0) (3 g) in dichloromethane (DCM) (250 ml). The reaction, which takes on a dark colouring, was allowed to stir at room temperature for 1.5 hours. Subsequently, the reaction was filtered over gooch, using as eluent mixture a hexane:ethyl acetate (AcOEt) solution in 9:1 ratio. The filtrate was concentrated under reduced pressure and the compound 2 was obtained in yield of 80%. [Corey E. J. and Suggs J. W. 1975]

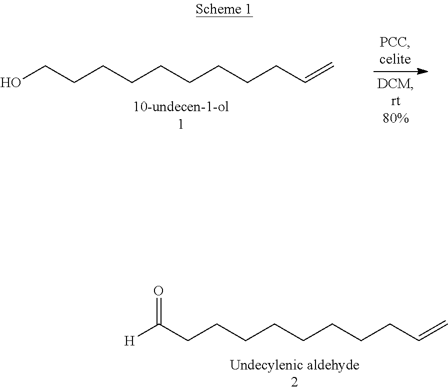

Oil (80% yield). ¹H NMR (CDCl₃, 400 MHz): δ (ppm)=9.76 (s, 1H, RCHO), 5.87-5.73 (m, 1H, RCH═CH₂), 4.98 (d, J=17.1 Hz, 1H, RCH═CH₂), 4.92 (d, J=10.2 Hz, 1H, RCH═CH₂), 2.07-1.98 (m, 2H, RCH₂CHO), 1.68-1.54 (m, 2H, RCH₂CH═CH₂), 1.43-1.21 (m, 12H, CH₂). ¹³C-NMR (CDCl₃, 100 MHz): δ (ppm)=202.98, 139.15, 114.17, 43.91, 33.77, 29.29, 29.25, 29.14, 29.03, 28.88, 22.07.

ESI-HRMS (positive) m/z: [M+Na]⁺ calculated for C₁₁H₂₀ONa 191.29; it was found: 191.29

Synthesis of compound 3: resorcinol (MERCK 203-585-2) (38 mmol, 4.18 g), previously crushed with mortar and pestle, was added to a solution of ethanol (EtOH) (16.25 ml) and hydrochloric acid (37% HCl) (5.41 ml). After 30 minutes, the solution takes on a white colouring, and undecylenic aldehyde 2 (38 mmol, 6.40 g.) previously solubilized in EtOH (10.58 ml) is added. Subsequently, the reaction was allowed to stir and reflux for 24 hours at 70° C. Subsequently, the reaction was brought to room temperature and concentrated under reduced pressure. The residue was purified by flash chromatography column using DCM:Methanol (MeOH) in 95:5 ratio as eluent mixture. The compound 3 was obtained in yield of 70%. [Thoden van Velzen E. U. et al. 1995]

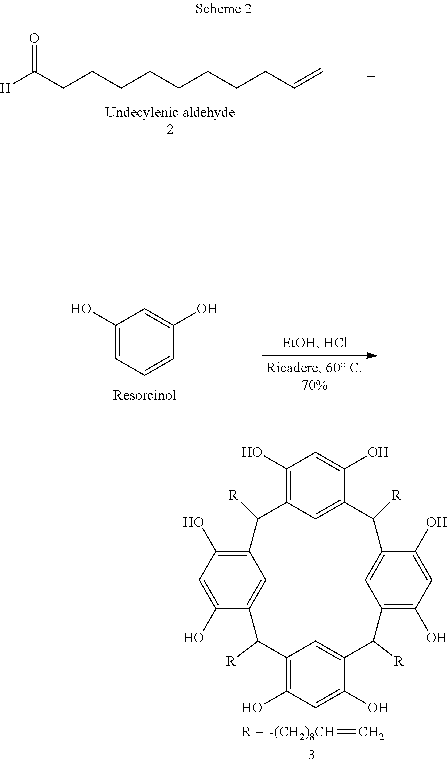

Brown powder (70% yield); p.f. 290±0.5° C. ¹H NMR (CDCl₃, 400 MHz): δ (ppm)=9.60 (br s, 8H, ArOH), 7.21 (s, 4H, ArHext.), 6.11 (s, 4H, ArHint.), 5.88-5.74 (m, 4H, RCH═CH₂), 4.99 (dd, J=17.1, 1.4 Hz, 4H, RCH═CH₂), 4.95 (d, J=10.1 Hz, 4H, RCH═CH₂), 4.30 (pseudo t, J=7.2 Hz, 4H, ArCHAr), 2.44-2.09 (m, 8H, RCH₂CHAr₂), 2.12-1.95 (m, 8H, RCH₂CH═CH₂), 1.45-1.20 (m, 48H, CH₂). ¹³C NMR (CDCl₃, 100 MHz): δ (ppm)=139.20, 114.15, 33.85, 29.71, 29.52, 29.17, 28.99, 27.99.

ESI-HRMS (positive) m/z: [M+Na]⁺ calculated for—C₆₈H₉₆O₈Na 1063. 69974; it was found 1063.70010.

Synthesis of compound 4: potassium carbonate (K₂CO₃) (19.2 mmol, 2.65 g) and methylbromoacetate (BrCH₂COOCH₃) (9.6 mmol, 1.47 g) (ratio of starting substrate to reagents 1:20:10) were added to a solution of resorcarene 3 (0.96 mmol, 1 g) in acetonitrile (ACN) (131.5 ml). The reaction was allowed to stir and reflux for 24 hours at 82° C. The solution was then evaporated under reduced pressure to remove ACN in excess and dissolved in DCM. The obtained organic phase was washed once with a 1 N HCl solution (70 ml) and twice with a saturated sodium chloride (NaCl) solution (140 mL). Finally, it was dehydrated with anhydrous sodium sulfate (Na₂SO₄) and concentrated under reduced pressure. The residue was allowed to stir 12 hours in MeOH, the precipitate was vacuum filtered obtaining the compound 4 in yield of 77%.

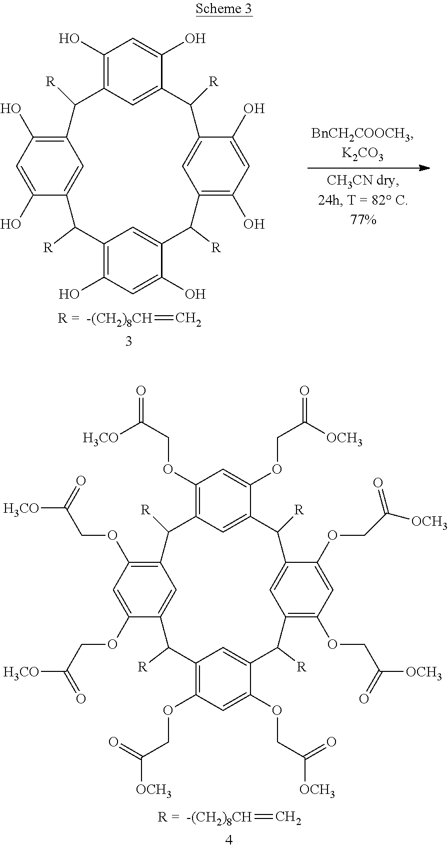

White powder (77% yield) p.f. 268±0.5° C. ¹H NMR (CDCl₃, 400 MHz): δ (ppm)=6.60 (s, 4H, ArHext.), 6.20 (s, 4H, ArHint.), 5.86-5.71 (m, 4H, RCH═CH₂), 4.96 (d, J=17.1 Hz, 4H, RCH═CH₂), 4.90 (dd, J=10.2, 0.9 Hz, 4H, RCH═CH₂), 4.58 (t, J=7.4 Hz, 4H, ArCHAr), 4.21

(s, 16H, ArOCH₂CO), 3.68 (s, 24H, CH₃OCOR), 1.99-1.88 (m, 8H, RCH₂CH═CH₂), 1.82-1.70 (m, 8H, RCH₂CHAr₂), 1.34-1.10 (m, 48H, CH₂). ¹³C NMR (CDCl₃, 100 MHz): δ (ppm)=169.94, 154.60, 139.35, 128.62, 126.68, 114.20, 100.87, 67.26, 52.05, 35.81, 34.63, 33.96, 30.08, 29.82, 29.80, 29.36, 29.11, 28.17.

ESI-HRMS (positive) m/z: [M+Na]⁺ calculated for C₉₂H₁₂₈O₂₄Na 1640.9537; it was found 1640.8669.

Synthesis of compound 5: 1-dodecanthiol (14.09 mmol, 2.85 g) and 9-borabicyclo[3.3.1.]nonane (BBN) (24.75 mmol, 3 g) were added at 0° C. to a solution of resorcarene 4 (0.618 mmol, 1 g) in tetrahydrofuran (THF) (53.74 ml). The reaction was allowed to stir for 12 hours at room temperature. Subsequently, the solvent was evaporated under reduced pressure and the residue was purified by crystallization in MeOH. The compound 5 was obtained in yield of 82%. [Thoden van Velzen E. U. et al. 1995]

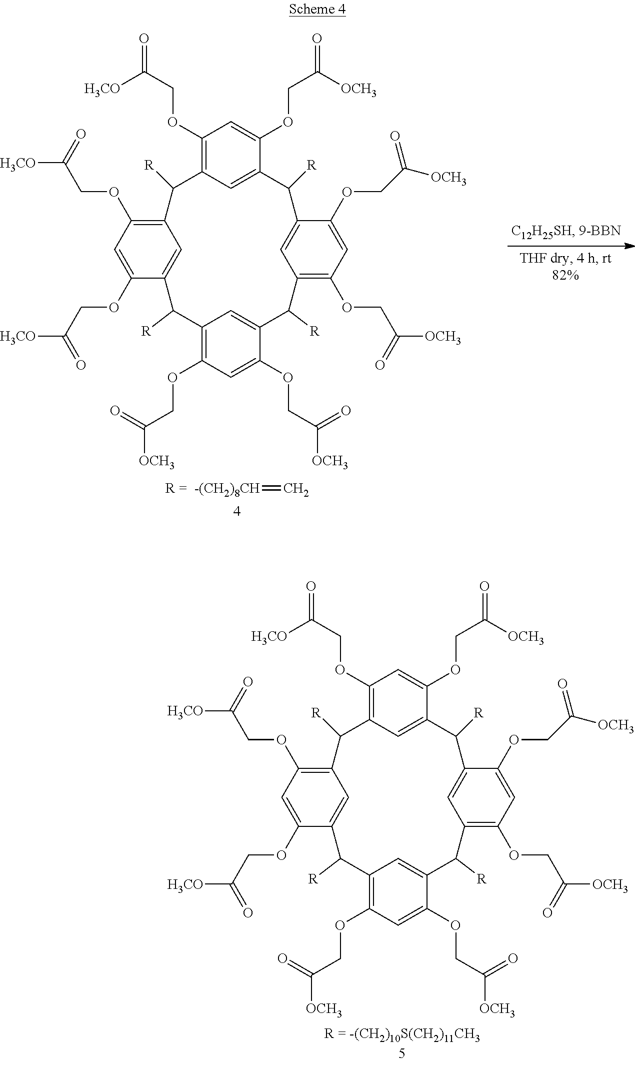

White powder (82% yield); p.f. 290±0.5° C. ¹H NMR (CDCl₃, 400 MHz): δ (ppm)=6.59 (s, 4H, ArHext.), 6.20 (s, 4H, ArHint.), 4.57 (t, J=7.4 Hz, 4H, ArCHAr), 4.27 (s, 16H, ArOCH₂CO), 3.75 (s, 24H, CH₃OCOR), 2.53-2.39 (m, 16H, —CH₂—S—CH₂—), 1.92-1.75 (m, 8H, CH₂), 1.67-1.46 (m, 16H, CH₂), 1.39-1.19 (m, 128H, CH₂), 0.87 (t, J=6.7 Hz, 12H, CH₃). ¹³C NMR (CDCl₃, 100 MHz): δ (ppm)=169.94, 154.61, 128.62, 126.68, 100.89, 67.27, 52.05, 35.82, 34.63, 32.35, 32.05, 30.11, 29.96, 29.94, 29.91, 29.88, 29.83, 29.80, 29.77, 29.76, 29.69, 29.53, 29.49, 29.43, 29.21, 29.13, 28.20, 22.82, 14.26.

ESI-HRMS (positive) m/z: [M+H]⁺ calculated for C₁₄₀H₂₃₂O₂₄S₄ 2425.58109; it was found [M+Na]⁺ 2447.56295.

Synthesis of compound 6: Resorcarene 5 (0.124 mmol, 300 mg) was solubilized in THF (17.3 ml) and subsequently treated with an aqueous solution of 2M potassium hydroxide (KOH) (7.44 ml) for 4 hours at room temperature. The reaction was then acidified with 2M HCl and concentrated under reduced pressure. The obtained residue was washed with water and dried at 80° C. under vacuum. The compound 6 was obtained in yield of 93%. [Hua B. et al. 2016]

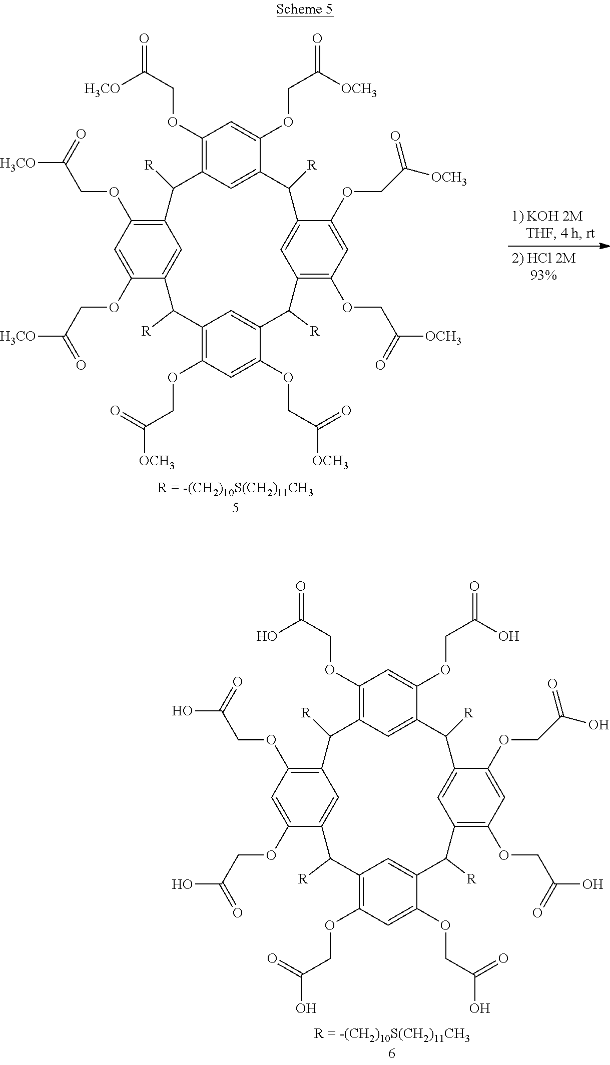

White powder (yield 93%); p.f. 270±0.5° C. ¹H NMR (CDCl₃:CD₃OD=98:2, 400 MHz): δ (ppm)=6.64 (s, 4H, ArHext.), 6.16 (s, 4H, ArHint.), 4.54 (t, J=7.1 Hz, 4H, ArCEAr), 4.48-3.98 (m, 16H, ArOCH₂CO), 2.54-2.34 (m, 16H, CH₂), 1.78 (br s, 8H, CH₂), 1.56-1.46 (m, 16H, CH₂), 1.37-1.10 (m, 128H, CH₂), 0.83 (t, J=6.6 Hz, 12H, CH₃). ¹³C NMR (CDCl₃:CD₃OD (98:2), 100 MHz): δ (ppm)=170.12, 154.45, 128.52, 126.59, 100.84, 67.14, 35.56, 34.61, 32.23, 31.95, 30.01, 29.84, 29.81, 29.77, 29.73, 29.70, 29.67, 29.65, 29.58, 29.42, 29.38, 29.31, 29.09, 29.01, 28.06, 22.72, 14.12. ESI-HRMS (negative) m/z: [M−2H]²⁻ calculated for C₁₃₂H₂₁₆O₂₄S₄ 1155.72094; it was found 1155.72196.

Synthesis of compound 7: Resorcarene 6 (0.043 mmol, 100 mg) was treated with 25-28% ammonium hydroxide (NH₄OH) solution at room temperature for 24 hours. Subsequently, the solution was concentrated under reduced pressure and the compound 7 was obtained in a quantitative yield. [Hua B. et al. 2016]

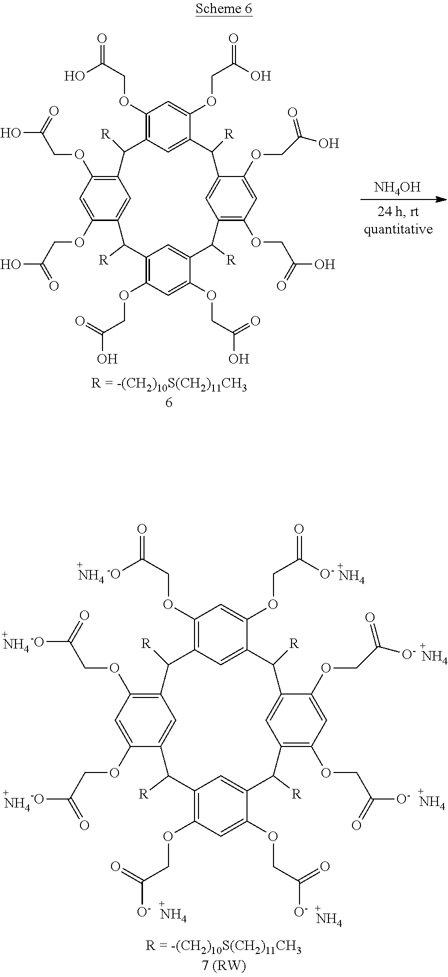

### Pink Powder (Quantitative Yield)

Modification of Magnetic Nanoparticles Decorated with Gold (Au@MNPs)

The RW compound was used to modify the surface of gold-decorated magnetic nanoparticles (Au@MNPs), allowing the realization of functionalized nanoparticles (RW/Au@MNPs). To this end, the Au@MNPs, once washed with water, were incubated in a rotating stirrer away from light, with a RW compound solution. Different concentrations of RW compound in water were evaluated, in a range comprised between 4 mM and 1.8 μM, (Ha, Solovyov, and Katz 2009) evaluating the stability thereof over time. The optimal concentration of RW compound was found to be 100 μM, and was chosen for realizing the immunosensor. The graphite screen printed (SPE) electrodes were used as electrochemical transducers for the realization of the immunosensor, depositing on the surface of the graphite working electrode, 20 μL of the RW/Au@MNPs solution. A magnet was used to prevent the loss of material during the washing processes that characterize the measurements. The system thus realized was then evaluated in the antibody loading capacity for atrazine (Ab-ATZ). Immobilization was achieved by depositing the antibody solution at different concentrations on the electrode surface. Subsequently, the surface was washed with phosphate buffer (PBS) and a 0.1 mg/mL albumin solution (BSA) was used to deactivate the RW compound molecules that did not react with the specific antibody, preventing the occurrence of any non-specific interactions in the incubation step with the antigen. The electrochemical characterization of the various phases involved in the realization of the immunosensor was carried out by differential pulse voltammetry (DPV) using as redox probe the iron-ferricyanide pair [Fe(CN)⁶]^{3-/4-}, an electrochemically active substance which is discharged on the surface of the electrode at a certain applied potential giving rise to a current signal. [Ramnani P. et al. 2016] Any modification made to the sensor surface, hindering the diffusion of the redox probe towards the electrode surface, results in the detected current intensity being lowered which is proportional to the quantity of substance that interacted on the electrode surface. [Ha J. M. et al. 2009; Ramnani P. et al. 2016]

### Immobilization of Ab-ATZ on RW/Au@AuMNPs

The electrodes were previously modified by deposition of Au@MNPs functionalized with a 100 μM solution of RW compound. In order to trace back the optimal concentration of antibody to be immobilized, a loading curve was constructed by observing the decrease in the current signal (ΔI) resulting after incubation of increasing quantities of antibody in the concentration range; 0.1-100 μg/ml (FIG. 1) in PB buffer (phosphate buffer) pH 7.4. The measurements were performed by differential pulse voltammetry in the presence of a solution 1.1 mM Fe(CN)₆^{3-/4-}, 100 mM KCl, in the potential range [−0.4-+0.6].

The antibody concentration chosen for realizing the immunosensor was equal to 20 μg/ml.

### Optimization of ATZ Incubation

In order to improve the interaction of Ab_{ATZ} with ATZ, different incubation times of an ATZ solution (1 ng/ml) on the surface of the realized immunosensor, in a range between 15 and 50 min, were evaluated by measuring the lowering of the intensity of current (FIG. 2).

The measurements were carried out in [Fe(CN)₆]^{3-/4-} in 1.1 mM deionized water (R=18 mOhm) in the range of potentials [−0.4-+0.6].

### ATZ Calibration

For immunosensor characterization, various concentrations of Atrazine were evaluated in a range comprised between 0.05 ng/mL and 10 ng/ml, with an incubation time of 30 minutes.

At the end of the process, the excess unbound antigen was removed by rinsing with 20 mM dilution phosphate buffer (PBS) pH 7.4 (FIGS. 3 and 4).

The measurements were carried out in [Fe(CN)₆]^{3-/4-} in 1.1 mM deionized water (R=18 mOhm) in the range of potentials [−0.4-+0.6].

The sensor obtained showed a sensitivity equal to 5.79 mL*μA/ng, a limit of detection (LOD) of 0.015 ng/mL and a linear dynamic range (linear range) 0.05-1 ng/mL, better analytical performance when compared to a comparison sensor obtained by chemical immobilization (Tab.1).

TABLE 1
Analytical performance obtained with RW-oriented
Ab immobilization and random immobilization.

| | Linear | Sensitivity | |
|---|---|---|---|
| Analytical | Range | (mL/ng)* | LOD |
| Performances | (ng/mL) | μA | (ng/mL) |
| Resorcarene | 0.05-1 | 5.7876 | 0.015 |
| Immobilization | | | |
| Random | 0.3-2 | 2.2215 | 0.035 |
| immobilization | | | |

### Description of the Random Immobilization Method

The graphite electrodes (SPE) were previously modified by depositing mercapto propionic acid (MPA)-functionalized Au@MNPs derived from incubating 10 μL of Au@MNPs in a 230 mM MPA solution in PBS pH7.4.

The MPA carboxyl groups were subsequently activated by treatment with 1:1 EDC/NHS at 4 mM concentration for 15 min. Once the excess reagent was rinsed with MES buffer pH 5.4, the surface was incubated with a 20 μg/mL solution of Anti-Atrazine Antibody (AbATZ) for 30 minutes and subsequently rinsed by PBS buffer. The electrode is then rinsed in PBS and incubated for 20 minutes with 1 M aqueous ethanolamine solution to deactivate the activated sites that have not reacted with AbATZ.

The measurements were carried out in FeCN₆^{3-/4-}1.1 mM 100 mM KCl cell in deionized water (R=18 mOhm) in the range of potentials [−0.4; +0.6]V using a graphite counter electrode and a calomel reference electrode (SCE). The electrochemical experiments were carried out using the Palmsens potentiostat.

As shown in FIG. 5, the curve concerning the oriented immobilization of the AbATZ thanks to the presence of the RT linker—with the same concentration used—is higher than the random chemical immobilization, to the advantage of the subsequent Ab-Ag interaction phase.

### BIBLIOGRAPHY

Chen H. et al. Talanta. 2008a; 75(1):99-103.
Chen H. Non-enzymatic glucose sensor and preparation method thereof. China Patent n. CN109342529B, 2008b;
Corey E. J. and Suggs J. W. Tetrahedron letters. 1975; 16(31):2647-2650.
Demirkol D. O. et al. RSC Advances. 2014; 4(38):19900-19907.
Ha J. M. et al. Langmuir. 2009; 25(18):10548-53.
Hua B. et al. New Journal of Chemistry. 2016; 40(5):4756-4760.
Jung Sung Ouk and Lee Soo Suk. Electrochemical sensor for detecting biomolecule, method for preparing the same, and method for detecting biomolecule using the electrochemical sensor. Korean Patent n. KR100773543B1, 2007;
Kim T-S. Calixcrown derivatives, a process for the preparation thereof, a self-assembled mono-layer of the calixcrown derivatives prepared by using the same and a process for immobilizing a protein mono-layer by using the self-assembled mono-layer of the calixcrown derivatives. Patent n. EP1110964A1, 2002;
Lee Y. et al. Proteomics. 2003; 3(12):2289-304.
Quaglio D. et al. Chemistry. 2020; 26(38):8400-8406.
Thoden van Velzen E. U. et al. Synthesis. 1995; 1995(08):989-997.
Ramnani P. et al. Chemosphere. 2016; 143:85-98.
Soler M. et al. Sensors. 2014; 14(2):2239-2258.
Sonmez B. et al. RSC Advances. 2014; 4(108):62895-62902.
Šustrová B. et al. Electroanalysis. 2010; 22(17-18):2051-57.

1. A compound formula (I): 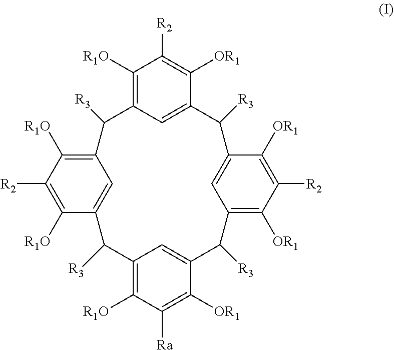
wherein:
R₁ is selected from C₂₋₆alkyl, (CH₂)ₙC(═O)OX, SO₃X, PO(OX)₂ and (CH₂)₂OH, wherein n is an integer from 1 to 5;
R₂ is selected from hydrogen, [heterocycle]-C(═O)OX, CH₂-[heterocycle]-C(═O)OX, CH₂SO₃X, CH₂PO(OX)₂; preferably said [heterocycle] is selected from piperidine, pyrrolidine or piperazine;
R₃ is selected from
a saturated linear aliphatic group containing sulfur of molecular formula (CH₂)ₘSH or (CH₂)ₘS(CH₂)ₘCH₃ wherein m is independently at each occurrence an integer from 5 to 11; or
an aromatic group of molecular formula C₆H₄—SH, C₄H₃S, C₆H₄—N₂Y;
X is independently selected from positively charged atoms or groups comprising: Na⁺, K⁺, NH₄⁺;
Y is selected from negatively charged atoms or groups comprising: Cl⁻, BF₄⁻.

2. The compound according to claim 1, wherein: R₁ is CH₂C(═O)OX and/or R₂ is hydrogen and/or R₃ is a saturated linear aliphatic group containing sulfur of molecular formula (CH₂)₁₀S(CH₂)₁₁CH₃, preferably X is NH₄⁺.

3. An electrode for the electrochemical detection of an analyte in a biological sample, wherein the electrode is a printed miniaturized carbon-based electrode comprising magnetic nanoparticles, said magnetic nanoparticles comprising on their surface the compound of formula (I) according claim 1.

4. The electrode for the electrochemical detection of an analyte in a biological sample according to claim 3, wherein:
the printed miniaturized carbon-based electrode is a graphite, graphene, carbon nanotubes or carbon fibers electrode; and/or
the magnetic nanoparticles are gold-coated magnetic nanoparticles (Au@MNPs).

5. The electrode for the electrochemical detection of an analyte in a biological sample according to claim 3, wherein the compound of formula (I) is non-covalently linked to at least one antibody able to detect to said analyte.

6. A biosensor for the electrochemical detection of an analyte in a biological sample comprising an electrode according to claim 3 as the working electrode, a reference electrode and a counter-electrode.

7. The biosensor for the electrochemical detection of an analyte in a biological sample according to claim 6, wherein the compound of formula (I) is non-covalently linked to at least one antibody able to detect atrazine (ATZ).

8. A method for the production of a biosensor for the electrochemical detection of an analyte in a biological sample according to claim 6, comprising the following steps:
a) adding magnetic nanoparticles to a solution comprising the compound of formula (I), so that at least one compound of formula (I) is bound on the surface of the nanoparticles;
b) adding the resulting solution on the surface of a printed miniaturized electrode and evaporating the liquid phase of the solution, preferably in the presence of a magnet located below the electrode;
c) adding a solution comprising at least one antibody specific for the analyte on the surface of the electrode obtained from step (b) and incubating for sufficient time allowing the formation of a non-covalent bond among said antibody and the compound of formula (I).

9. A method for the electrochemical detection of an analyte in a biological sample comprising the following steps:
a) contacting the biological sample with the biosensor according to claim 6, so that the binding of the antibody to the analyte on the working electrode results in a change in the passage of current between the working electrode and the counter electrode;
b) detecting the current change generated in step a) and measuring the presence or quantity of the analyte in the sample by comparison with a calibration curve.

10. An electrode for the electrochemical detection of an analyte in a biological sample, wherein the electrode is a printed miniaturized carbon-based electrode comprising gold-coated nanoparticles, said nanoparticles comprising on their surface a compound of formula (II): 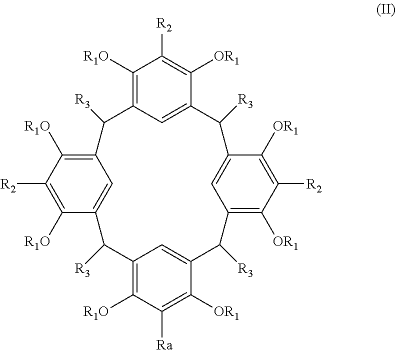
wherein
R₁ is selected from hydrogen, alkyl, (CH₂)ₙC(═O)OX, SO₃X, PO(OX)₂, (CH₂)₂OH, wherein n is an integer from 1 to 5;
R₂ is selected from hydrogen, [heterocycle]-C(═O)OX, CH₂-[heterocycle]-C(═O)OX, CH₂SO₃X, CH₂PO(OX)₂; preferably said [heterocycle] is piperidine, pyrrolidine or piperazine;
R₃ is selected from
a saturated linear aliphatic group containing sulfur of molecular formula (CH₂)ₘSH or (CH₂)ₘS(CH₂)ₘCH₃, wherein m is independently at each occurrence an integer from 5 to 11; or
an aromatic group of molecular formula C₆H₄—SH, C₄H₃S, C₆H₄—N₂Y;
X is independently selected from positively charged atoms or groups comprising: Na⁺, K⁺, NH₄⁺;
Y is selected from negatively charged atoms or groups comprising: Cl⁻, BF₄⁻.
wherein said compound of formula (II) is non-covalently bound to at least one antibody able to detect said analyte.
